(19) United States
(12) Patent Application Publication
(10) Pub. No.: US 2010/0049099 A1
(43) Pub. Date: Feb. 25, 2010

(54) METHOD AND SYSTEM FOR POSITIONING AN ENERGY SOURCE

(75) Inventors: Hira V. THAPLIYAL, Los Altos, CA (US); David A. GALLUP, Alameda, CA (US); James W. ARENSON, Woodside, CA (US)

Correspondence Address: VytronUS, Inc. & Townsend and Townsend nd Crew LLP;Joint CN, Two Embarcadero Center, Eighth Floor, San Francisco, CA, 94111, US

(73) Assignee: VytronUS, Inc., Sunnyvale, CA (US)

(21) Appl. No.: 12/505,326

(22) Filed: Jul. 17, 2009

Related U.S. Application Data
(60) Provisional application No. 61/082,059, filed on Jul. 18, 2008.

(51) Int. Cl. A61N 7/00; A61B 18/18; A61B 18/04; A61B 18/20

(52) U.S. Cl. 601/2; 606/33; 606/27; 606/14; 606/21

(57) ABSTRACT

An ablation system for treating atrial fibrillation in a patient comprises an inner shaft having proximal and distal ends as well as a lumen therebetween. A distal tip assembly is adjacent the inner shaft distal end, and the distal tip assembly comprises an energy source and a sensor. The energy source is adapted to deliver energy to a target tissue so as to create a zone of ablation in the target tissue. This blocks abnormal electrical activity and thus reduces or eliminates atrial fibrillation in the patient. The system also has an outer shaft with proximal and distal ends, and a lumen therebetween. The inner shaft is slidably disposed in the outer shaft lumen, and the inner shaft is rotatable, bendable and linearly slidable relative to the outer shaft. The outer shaft is rotatable, bendable and linearly slidable relative to the target tissue.

### CROSS-REFERENCES TO RELATED APPLICATIONS

This application is a non-provisional of and claims the benefit of priority of U.S. Provisional Patent Application No. 61/082,059 (Attorney Docket No. 027680-000600US) filed Jul. 18, 2008, the entire contents of which are incorporated herein by reference.

### BACKGROUND OF THE INVENTION

1. Field of the Invention

The present invention relates generally to medical devices, systems and methods, and more specifically to improved devices, systems and methods for creating an ablation zone in tissue. The device may be used to treat atrial fibrillation.

The condition of atrial fibrillation (AF) is characterized by the abnormal (usually very rapid) beating of left atrium of the heart which is out of synch with the normal synchronous movement (“normal sinus rhythm”) of the heart muscle. In normal sinus rhythm, the electrical impulses originate in the sino-atrial node (“SA node”) which resides in the right atrium. The abnormal beating of the atrial heart muscle is known as fibrillation and is caused by electrical impulses originating instead in the pulmonary veins (“PV”) [Haissaguerre, M. et al., Spontaneous Initiation of Atrial Fibrillation by Ectopic Beats Originating in the Pulmonary Veins, New England J Med., Vol. 339:659-666].

There are pharmacological treatments for this condition with varying degrees of success. In addition, there are surgical interventions aimed at removing the aberrant electrical pathways from the PV to the left atrium (“LA”) such as the Cox-Maze III Procedure [J. L. Cox et al., The development of the Maze procedure for the treatment of atrial fibrillation, Seminars in Thoracic & Cardiovascular Surgery, 2000; 12: 2-14; J. L. Cox et al., Electrophysiologic basis, surgical development, and clinical results of the maze procedure for atrial flutter and atrial fibrillation, Advances in Cardiac Surgery, 1995; 6: 1-67; and J. L. Cox et al., Modification of the maze procedure for atrial flutter and atrial fibrillation. II, Surgical technique of the maze III procedure, Journal of Thoracic & Cardiovascular Surgery, 1995; 2110:485-95]. This procedure is shown to be 99% effective [J. L. Cox, N. Ad, T. Palazzo, et al. Current status of the Maze procedure for the treatment of atrial fibrillation, Seminars in Thoracic & Cardiovascular Surgery, 2000; 12: 15-19] but requires special surgical skills and is time consuming.

There has been considerable effort to copy the Cox-Maze procedure for a less invasive percutaneous catheter-based approach. Less invasive treatments have been developed which involve use of some form of energy to ablate (or kill) the tissue surrounding the aberrant focal point where the abnormal signals originate in the PV. The most common methodology is the use of radio-frequency (“RF”) electrical energy to heat the muscle tissue and thereby ablate it. The aberrant electrical impulses are then prevented from traveling from the PV to the atrium (achieving conduction block within the heart tissue) and thus avoiding the fibrillation of the atrial muscle. Other energy sources, such as microwave, laser, and ultrasound have been utilized to achieve the conduction block. In addition, techniques such as cryoablation, administration of ethanol, and the like have also been used.

There has been considerable effort in developing catheter based systems for the treatment of AF using radiofrequency (RF) energy. One such method is described in U.S. Pat. No. 6,064,902 to Haissaguerre et al. In this approach, a catheter is made of distal and proximal electrodes at the tip. The catheter can be bent in a J shape and positioned inside a pulmonary vein. The tissue of the inner wall of the PV is ablated in an attempt to kill the source of the aberrant heart activity. Other RF based catheters are described in U.S. Pat. No. 6,814,733 to Schwartz et al., U.S. Pat. No. 6,996,908 to Maguire et al., U.S. Pat. No. 6,955,173 to Lesh, and U.S. Pat. No. 6,949,097 to Stewart et al.

Another source used in ablation is microwave energy. One such device is described by Dr. Mark Levinson [(Endocardial Microwave Ablation: A New Surgical Approach for Atrial Fibrillation; The Heart Surgery Forum, 2006] and Maessen et al. [Beating heart surgical treatment of atrial fibrillation with microwave ablation. Ann Thorac Surg 74: 1160-8, 2002]. This intraoperative device consists of a probe with a malleable antenna which has the ability to ablate the atrial tissue. Other microwave based catheters are described in U.S. Pat. No. 4,641,649 to Walinsky; U.S. Pat. No. 5,246,438 to Langberg; U.S. Pat. No. 5,405,346 to Grundy et al.; and U.S. Pat. No. 5,314,466 to Stem et al.

Another catheter based method utilizes the cryogenic technique where the tissue of the atrium is frozen below a temperature of −60 degrees C. This results in killing of the tissue in the vicinity of the PV thereby eliminating the pathway for the aberrant signals causing the AF [A. M. Gillinov, E. H. Blackstone and P. M. McCarthy, Atrial fibrillation: current surgical options and their assessment, Annals of Thoracic Surgery 2002; 74:2210-7]. Cryo-based techniques have been a part of the partial Maze procedures [Sueda T., Nagata H., Orihashi K. et al., Efficacy of a simple left atrial procedure for chronic atrial fibrillation in mitral valve operations, Ann Thorac Surg 1997; 63:1070-1075; and Sueda T., Nagata H., Shikata H. et al.; Simple left atrial procedure for chronic atrial fibrillation associated with mitral valve disease, Ann Thorac Surg 1996; 62: 1796-1800]. More recently, Dr. Cox and his group [Nathan H., Eliakim M., The junction between the left atrium and the pulmonary veins, An anatomic study of human hearts, Circulation 1966; 34:412-422, and Cox J. L., Schuessler R. B., Boineau J. P., The development of the Maze procedure for the treatment of atrial fibrillation, Semin Thorac Cardiovasc Surg 2000; 12:2-14] have used cryoprobes (cryo-Maze) to duplicate the essentials of the Cox-Maze III procedure. Other cryo-based devices are described in U.S. Pat. Nos. 6,929,639 and 6,666,858 to Lafintaine and U.S. Pat. No. 6,161,543 to Cox et al.

More recent approaches for the AF treatment involve the use of ultrasound energy. The target tissue of the region surrounding the pulmonary vein is heated with ultrasound energy emitted by one or more ultrasound transducers. One such approach is described by Lesh et al. in U.S. Pat. No. 6,502,576. Here the catheter distal tip portion is equipped with a balloon which contains an ultrasound element. The balloon serves as an anchoring means to secure the tip of the catheter in the pulmonary vein. The balloon portion of the catheter is positioned in the selected pulmonary vein and the balloon is inflated with a fluid which is transparent to ultrasound energy. The transducer emits the ultrasound energy which travels to the target tissue in or near the pulmonary vein and ablates it. The intended therapy is to destroy the electrical conduction path around a pulmonary vein and thereby restore the normal sinus rhythm. The therapy involves the creation of a multiplicity of lesions around individual pulmonary veins as required. The inventors describe various configurations for the energy emitter and the anchoring mechanisms.

Yet another catheter device using ultrasound energy is described by Gentry et al. [Integrated Catheter for 3-D Intracardiac Echocardiography and Ultrasound Ablation, IEEE Transactions on Ultrasonics, Ferroelectrics, and Frequency Control, Vol. 51, No. 7, pp 799-807]. Here the catheter tip is made of an array of ultrasound elements in a grid pattern for the purpose of creating a three dimensional image of the target tissue. An ablating ultrasound transducer is provided which is in the shape of a ring which encircles the imaging grid. The ablating transducer emits a ring of ultrasound energy at 10 MHz frequency. In a separate publication [Medical Device Link, Medical Device and Diagnostic Industry, February 2006], in the description of the device, the authors assert that the pulmonary veins can be imaged.

While these devices and methods are promising, improved devices and methods for creating a heated zone of tissue, such as an ablation zone are needed. Furthermore, it would also be desirable if such devices could create single or multiple ablation zones to block abnormal electrical activity in the heart in order to lessen or prevent atrial fibrillation. It would also be desirable if such devices could be used in the presence of blood or other body tissues without coagulating or clogging up the ultrasound transducer. Such devices and methods should be easy to use, minimally invasive, cost effective and simple to manufacture.

2. Description of Background Art

Other devices based on ultrasound energy to create circumferential lesions are described in U.S. Pat. Nos. 6,997,925; 6,966,908; 6,964,660; 6,954,977; 6,953,460; 6,652,515; 6,547,788; and 6,514,249 to Maguire et al.; U.S. Pat. Nos. 6,955,173; 6,052,576; 6,305,378; 6,164,283; and 6,012,457 to Lesh; U.S. Pat. Nos. 6,872,205; 6,416,511; 6,254,599; 6,245,064; and 6,024,740; to Lesh et al.; U.S. Pat. Nos. 6,383,151; 6,117,101; and WO 99/02096 to Diederich et al.; U.S. Pat. No. 6,635,054 to Fjield et al.; U.S. Pat. No. 6,780,183 to Jimenez et al.; U.S. Pat. No. 6,605,084 to Acker et al.; U.S. Pat. No. 5,295,484 to Marcus et al.; and WO 2005/117734 to Wong et al.

### BRIEF SUMMARY OF THE INVENTION

The present invention relates generally to medical devices and methods, and more specifically to medical devices and methods used to deliver energy to tissue as a treatment for atrial fibrillation and other medical conditions.

In a first aspect of the present invention an ablation system for treating atrial fibrillation in a patient comprises an elongate inner shaft having a proximal end, a distal end and lumens therebetween. The system also has a distal tip assembly adjacent the distal end of the inner shaft. The distal tip assembly comprises an energy source and a sensor. The energy source is adapted to deliver energy to a target tissue so as to create a zone of ablation in the target tissue that blocks abnormal electrical activity thereby reducing or eliminating the atrial fibrillation in the patient. The system also has an elongate outer shaft having a proximal end, a distal end and a lumen therebetween. The inner shaft is slidably disposed in the outer shaft lumen. The inner shaft is rotatable, bendable and linearly slidable relative to the outer shaft, and the outer shaft is rotatable, bendable and linearly slidable relative to the target tissue.

The distal tip assembly may comprise an outer housing with the energy source and the sensor disposed therein. The outer housing may have an open end and may have a plurality of slots on one end forming a castellated region.

The energy source may be recessed from a distal end of the housing such that the energy source does not contact the target tissue or surrounding fluids such as blood when the energy source delivers energy. The energy source may comprise an ultrasound transducer. The energy source may deliver one of radiofrequency energy, microwaves, photonic energy, thermal energy, and cryogenic energy. The energy source may deliver the energy in a beam that is 65 to 115 degrees relative to a surface of the target tissue. The zone of ablation may follow an arcuate or a linear path.

The sensor may detect gap distance between a surface of the target tissue and the energy source. The sensor also may be able to detect an angle between the energy source and a surface of the target tissue. The sensor may be able to determine characteristics of the target tissue such as its thickness. In some embodiments, the sensor comprises an ultrasound transducer. The energy source may also be an ultrasound transducer, and in some embodiments the ultrasound transducer serves as both the energy source and the sensor. The sensor may also comprise an infrared sensor or a radiofrequency sensor.

The sensor may comprise a positioning mechanism adjacent the distal end of the outer shaft. The positioning mechanism may be adapted to facilitate location of an anatomic structure and also adapted to anchor the ablation system to the anatomic structure. The positioning mechanism may also provide a visual, audible or tactile indication of ablation system position relative to the anatomic structure. The ablation system inner shaft may be rotatable around the positioning mechanism.

The positioning mechanism may be positionable in the outer shaft lumen and may be in a substantially linear configuration while disposed therein. The positioning mechanism may comprise a coil or a plurality of wires that are biased to flare radially outward when unconstrained. The positioning mechanism may be adapted to exert an outward biasing force against the anatomic structure, thereby anchoring the ablation system thereto. The target tissue may comprise a pulmonary vein and the positioning mechanism may be adapted to indicate the angle of entry of the inner shaft into the pulmonary vein.

The system may further comprise a guide catheter and the outer shaft may be slidably positioned therein. The target issue may comprise heart tissue, a pulmonary vein or tissue adjacent thereto. The inner or the outer shaft may comprise a braided portion or a spring coil. The system may also comprise an anchoring mechanism that is coupled with the ablation system and that is configured to stabilize the distal tip assembly. The anchoring mechanism may comprise an expandable member such as a balloon. The anchoring mechanism may also comprise a shapeable wire that may be coupled with the target tissue. The anchor may also have one or more tissue engaging barbs or hooks.

The system may further comprise a bending mechanism that is operably coupled with the inner shaft. In some embodiments, the bending mechanism may comprise a pull wire operably coupled adjacent the distal end of the inner shaft and wherein a portion of the pull wire is disposed along an outer surface of the inner shaft such that when the pull wire is actuated, the inner shaft deflects radially inward or outward relative to the portion of the pull wire outside of the inner shaft, and wherein the portion of the pull wire remains in a substantially linear configuration. The bending mechanism may comprise a first and a second pull wire. The first pull wire may be coupled with a distal region of the inner shaft and the second pull wire may be coupled with a proximal region of the inner shaft. The pull wires may be adapted to bend the inner shaft in two locations, a first bend and a second bend. The system may include an actuator that is disposed near a proximal end of the inner shaft and that is adapted to actuate the pull wires thereby bending the inner shaft and forming the first bend and the second bend along the inner shaft. The first and second bends may be in different planes.

The system may comprise a bending mechanism that is operably coupled with the outer shaft. The bending mechanism may comprise a first and a second pull wire. The first pull wire may be coupled with a distal region of the outer shaft and the second pull wire may be coupled with a proximal region of the outer shaft. The pull wires may be adapted to bend the outer shaft in two locations, a first bend and a second bend. The system may also have an actuator that may be disposed near a proximal end of the outer shaft and that may be adapted to actuate the pull wires thereby bending the inner shaft and forming the first bend and the second bend along the inner shaft. The first and the second bends may be in different planes.

In a second aspect of the present invention, a method for treating atrial fibrillation in a patient by ablating tissue comprises providing an ablation system comprising an outer shaft and an inner shaft. The inner shaft has a distal tip assembly that comprises an energy source and a sensor. The outer shaft is slidably disposed over at least a portion of the inner shaft. The distal tip assembly is positioned adjacent the tissue and the inner shaft or the outer shaft is manipulated so as to place the energy source in a desired position relative to the tissue. Energy is delivered from the energy source to the tissue and a partial or complete zone of ablation is created in the tissue, thereby blocking abnormal electrical activity and reducing or eliminating the atrial fibrillation.

The ablation system may further comprise a guide sheath and the method may include positioning a distal portion of the guide sheath across an atrial septum of the patient's heart. Positioning the system may comprise advancing the distal tip assembly intravascularly into the patient's heart.

The step of manipulating may comprise slidably moving the inner shaft relative to the outer shaft. Manipulating may also include rotating the inner shaft relative to the outer shaft or bending the inner shaft. Bending may also comprise bending the inner shaft in two or more locations that may lie in the same plane or in different planes. Bending may be accomplished by actuating one or more pull wires coupled to the inner shaft. The step of manipulating may also include slidably moving, rotating or bending the outer shaft. The outer shaft may be bent in two or more locations. The two or more bends may lie in the same or different planes. Bending of the outer shaft may be accomplished by actuating one or more pull wires that are coupled to the outer shaft. The step of manipulating may comprise rotating either the inner or the outer shaft in a first direction and rotating the inner or the outer shaft in a second direction opposite the first direction so as to reduce binding or torque buildup in the inner or the outer shaft. The step of manipulating may comprise moving the energy source so as to direct the energy from the energy source to the tissue in a raster pattern. The manipulating step may comprise synchronizing movement of the energy source with the patient's heart rate.

The energy source may comprise an ultrasound transducer and the step of delivering the energy may comprise delivering an ultrasound beam from the transducer to the tissue. The step of delivering the energy may comprise delivering one of radiofrequency energy, microwave energy, photonic energy, thermal energy, and cryogenic energy.

Creating the zone of ablation may comprise forming a linear ablation path or a circular ablation path which may encircle at least one pulmonary vein. The ablation system may further comprise a sensor and the method may comprise sensing characteristics of the tissue with the sensor. The sensor may comprise an ultrasound transducer and in some embodiments the energy source may comprise the same ultrasound transducer as the sensor. The method may further comprise switching modes between delivering energy from the ultrasound transducer and sensing with the ultrasound transducer. The sensed tissue characteristics may comprise position of the tissue relative to the energy source, such as gap distance between the tissue and a surface of the energy source. The position may comprise a relative angle between the energy source and the tissue. The tissue characteristics may comprise the tissue thickness and/or the depth of the ablated region.

The sensor may comprise a positioning mechanism and the method may include advancing the positioning mechanism from either the inner or the outer shaft into the tissue or tissue adjacent thereto. The positioning mechanism may facilitate locating the anatomical structure. The positioning mechanism may comprise a plurality of wires and the method may further comprise positioning the wires into a pulmonary vein while observing the shape and orientation of the wires.

The method may further comprise guiding the energy based on the tissue characteristics that are sensed. The method may also include maintaining the gap between the energy source and the tissue surface at a desired value. The tissue may comprise left atrial tissue, a pulmonary vein or tissue adjacent thereto. In some embodiments the method further comprises cooling the energy source or anchoring the distal tip assembly relative to the tissue. Anchoring may comprise coupling a wire with the tissue or expanding an expandable member coupled with the outer shaft, such as a balloon.

These and other embodiments are described in further detail in the following description related to the appended drawing figures.

### BRIEF DESCRIPTION OF THE DRAWINGS

FIG. 1 is a drawing of bending movement, rotating movement, and linear movement of the system of the preferred embodiments of the invention;

FIG. 2 is a drawing of the distal tip assembly of the system of the preferred embodiments of the invention;

FIG. 3 is a drawing of the first variation of the anchoring mechanism of the system of the preferred embodiments of the invention;

FIGS. 4A-4C are drawings of bending movement of the system of the preferred embodiments of the invention;

FIG. 5 is a drawing of a “Sheppard's hook” bending movement of the system of the preferred embodiments of the invention;

FIGS. 6A and 6B are drawings of a second variation of bending movement of the system of the preferred embodiments of the invention;

FIG. 7 is a drawing of a movement pattern;

FIG. 8 is a drawing of a Electrocardiogram (ECG) tracing of a cardiac cycle;

FIG. 9 is a drawing of the connector console of the preferred embodiments; and

FIGS. 10A-13 are drawings of a positioning mechanism of the system of the preferred embodiments of the invention.

### DETAILED DESCRIPTION OF THE INVENTION

The following description of the preferred embodiments of the invention is not intended to limit the invention to these embodiments, but rather to enable any person skilled in the art to make and use this invention.

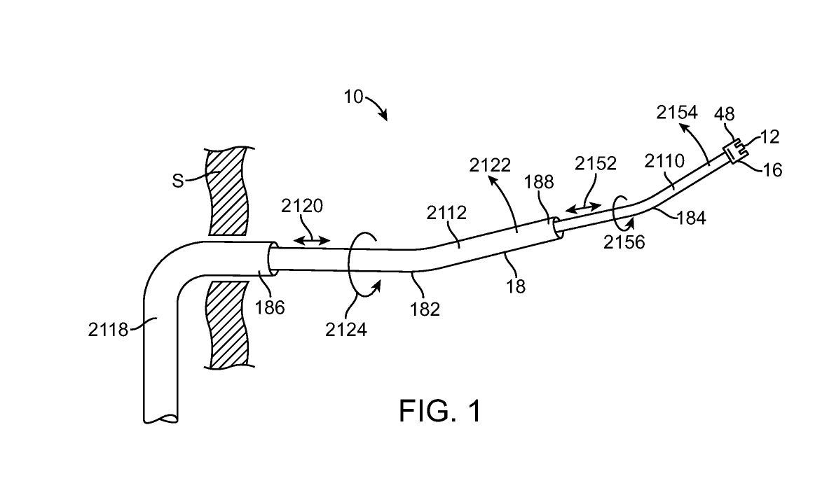

As shown in FIG. 1, the system 10 of the preferred embodiments includes an elongate member 18 and a distal tip assembly 48, which is coupled to the distal portion of the elongate member 18 and includes at least one of an energy source 12 and a sensor. The elongate member 18 functions to move and position the distal tip assembly 48, along with the energy source 12 and/or sensor. The elongate member 18 and the distal tip assembly 48 cooperatively function to direct the energy source 12 and/or the sensor towards a target tissue. The system 10 is preferably designed for positioning an energy source within a patient and delivering energy to tissue, more specifically, for delivering ablation energy to tissue, such as heart tissue, to create a conduction block—isolation and/or block of conduction pathways of abnormal electrical activity, which typically originate from the pulmonary veins in the left atrium for treatment of atrial fibrillation in a patient. The system 10, however, may be alternatively used with any suitable tissue in any suitable environment and for any suitable reason.

The Elongate Member. As shown in FIG. 1, the elongate member 18 of the preferred embodiments functions to move and position the distal tip assembly 48, and the energy source 12 and/or sensor within it. The elongate member 18 is preferably a catheter made of a flexible multi-lumen tube, but may alternatively be a cannula, tube or any other suitable elongate structure having one or more lumens. The elongate member 18 may also function to accommodate pull wires, fluids, gases, energy sources, electrical connections, therapy catheters, navigation catheters, pacing catheters, and/or any other suitable device or element.

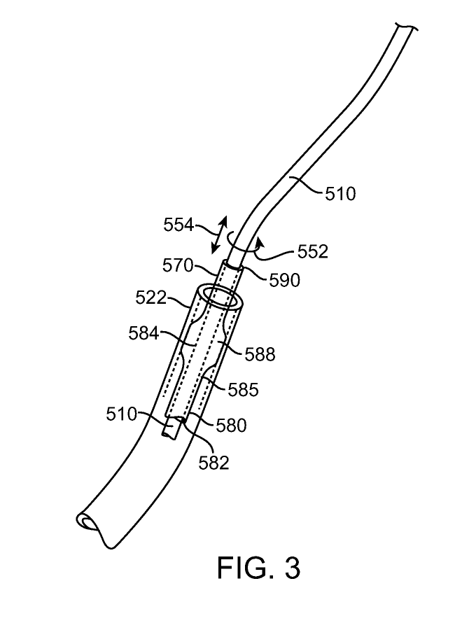

The elongate member is preferably one of several variations. In a first variation, as shown in FIG. 1, the elongate member 18 preferably includes a therapy catheter 2110, an outer catheter 2112, and a guide sheath 2118. The elongate member 18 may alternatively include a single catheter or any other suitable number of catheters and/or sheaths. The catheters are preferably arranged concentrically to one another, but may alternatively be arranged in any other suitable fashion. The therapy catheter 2110 is preferably slideably contained in the outer catheter 2112, and the therapy catheter 2110 and the outer catheter 2112 preferably form a conjoined set that can be freely moved axially in the guide sheath 2118. The outer catheter 2112 is preferably provided with at least three independent movements: axial movement 2120, rotational movement 2124, and bending movement 2122. The end portion 186 of the guide sheath 2118 preferably has a snug fit over the outer catheter 2112 so as to provide a grip on the outer catheter 2112 while it is performing rotation 2124. The anchoring mechanism, as shown in FIG. 3 and described below, is preferably used to fix the outer catheter 2112 with respect to the guide sheath 2118 while it rotates, bends, moves axially, or moves in any other suitable fashion. The outer catheter 2112 may be moved axially inside the guide sheath 2118 in a manner 2120. In addition, a portion of the outer catheter 2112 may be bent about a pivot point 182 in a manner 2122. Additionally, the outer catheter 2112 may bend in any suitable number of locations in addition to point 182.

Similarly to the outer catheter 2112, as shown in FIG. 1, the therapy catheter 2110 also is provided with at least three independent movements: axial movement 2152, rotational movement 2156, and bending movement 2154. The end portion 188 of the outer catheter 2112 preferably has a snug fit over the therapy catheter 2110 so as to provide a grip on the therapy catheter 2110 while it is performing rotation 2156. The therapy catheter 2110 may also be moved axially inside the outer catheter 2112 in a manner 2152. In addition, a portion of the therapy catheter 2110 may be bent about a pivot point 184 in a manner 2154. The therapy catheter 2110 may bend in any suitable number of locations in addition to point 184.

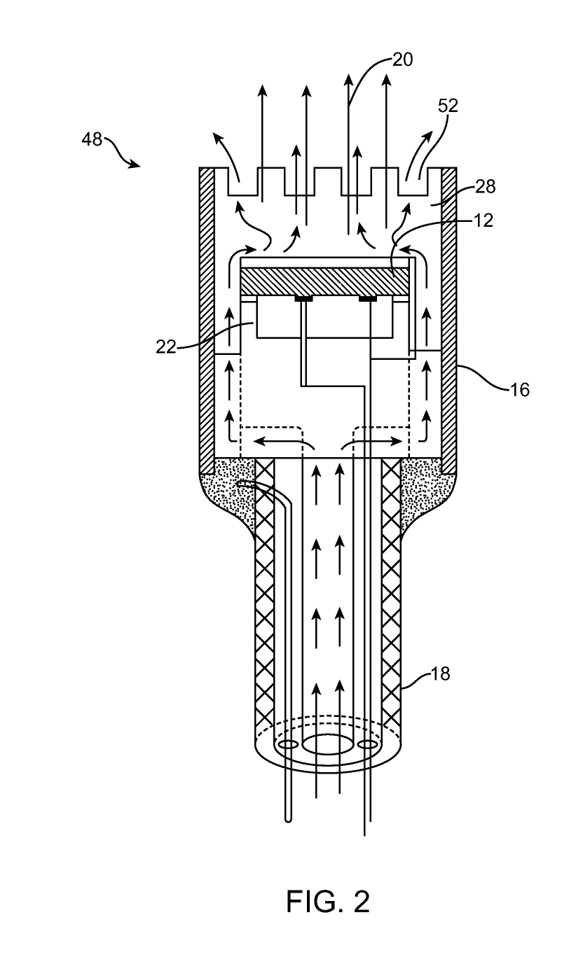

The Distal Tip Assembly. As shown in FIGS. 1 and 2, the distal tip assembly 48 of the preferred embodiments is coupled to the distal portion of the elongate member 18 and includes at least one of an energy source 12 and a sensor. As shown in FIG. 1, the distal tip assembly 48 preferably includes a housing 16 coupled to the energy source 12. The housing 16 preferably has an open, tubular shape, but may alternatively be a closed end housing that encloses the energy source 12. At least a portion of the closed end housing is preferably made of a material that is transparent to the energy beam 20, such as a material transparent to ultrasound energy, such as a poly 4-methyl, 1-pentene (PMP) material or any other suitable material. As shown in FIG. 2, the open tubular housing preferably has a “castle head” configuration such that the housing defines a plurality of slots 52. The slots 52 function to provide exit ports for the flowing fluid 28. When the front tip of the distal tip assembly 48 is in contact with or adjacent to the tissue or other structures during the use of the system 10, the slots 52 function to maintain the flow of the cooling fluid 28 past the energy source 12 and along the surface of the tissue. The fluid flow lines 30 flow along the grooves in the backing 22, bathe the energy source 12, form a fluid column and exit through the slots 52 at the castle head housing 16. In the closed end housing, the housing preferably defines apertures such as small holes towards the distal end of the housing 16. These holes provide for the exit path for the flowing fluid. The apertures may be a grating, screen, holes, drip holes, weeping structure or any of a number of suitable apertures. Alternatively, the closed end housing may not define apertures to allow the exit of the fluid but rather, the housing contains the fluid within the housing and recycles the fluid past the energy source 12.

The housing 16 of the distal tip assembly 48, further functions to provide a barrier between the face of the energy source 12 and the blood residing in the patient, such as in the atrium of the heart. If fluid flow is not incorporated, and the transducer face is directly in contact with blood, the blood will coagulate on the surface of the energy source 12. Additionally, there is a possibility of forming a blood clot at the interface of the energy source 12 and the surrounding blood. Because the energy source is recessed from the distal end of the housing and because the flow of the cooling fluid 28 keeps the blood from contacting the energy source 12, blood clot formation on the energy source is avoided. The fluid flow rate is preferably 1 ml per minute or higher (e.g. 10 ml per minute), but may alternatively be any other suitable flow rate to maintain the fluid column, keep the separation between the blood and the face of the energy source 12, cool the energy source 12, and/or cool the tissue 276. Additional details about housing 16 and the components therein are disclosed in greater detail in U.S. patent application Ser. No. 12/480,256 (Attorney Docket No. 027680-000310US), Ser. No. 12/483,174 (Attorney Docket No. 027680-000410US), and Ser. No. 12/482,640 (Attorney Docket No.027680-000510US), the entire contents of each incorporated herein by reference.

The Energy Source. As shown in FIG. 2, the energy source 12 of the preferred embodiments functions to provide a source of ablation energy and emits an energy beam 20. The energy source 12 is preferably an ultrasound transducer that emits an ultrasound beam, but may alternatively be any suitable energy source that functions to provide any suitable source of ablation energy. Some examples of suitable sources of ablation energy include radio frequency (RF) energy, microwaves, photonic energy, and thermal energy. The therapy could alternatively be achieved using cooled fluids (e.g., cryogenic fluid). The distal tip assembly 48 preferably includes a single energy source 12, but may alternatively include any suitable number of energy sources 12. The ultrasound transducer is preferably made of a piezoelectric material such as PZT (lead zirconate titanate) or PVDF (polyvinylidine difluoride), or any other suitable ultrasound beam emitting material. The transducer may further include coating layers such as a thin layer of a metal. Some suitable transducer coating metals may include gold, stainless steel, nickel-cadmium, silver, and a metal alloy.

The Sensor. The distal tip assembly 48 of the preferred embodiments also includes a sensor that functions to detect the gap (namely, the distance of the tissue surface from the energy source 12); the angle of the distal tip assembly 48, energy source 12, and/or sensor itself with respect to the tissue; the thickness of the tissue targeted for ablation; the characteristics of the ablated tissue; and any other suitable parameter or characteristic. By detecting this information, the information from the sensor preferably guides the therapy provided by the ablation of the tissue and provides information as to where to position the system, at what position to have the energy source with respect to the distal tip assembly in order to maintain a proper gap distance, and at what settings at which to use the energy source 12 and any other suitable elements. In response to the information detected by the sensor, the elongate member 18 and any suitable combination of outer catheters 2112 and therapy catheters 2110 can be moved axially, rotationally, in a bending movement, or in any suitable combination of movements thereof in order to move and position the distal tip assembly 48, the energy source 12, and/or the sensor within the patient, to maintain a sufficient gap distance and provide the proper characteristics and qualities desired of the ablated tissue. The sensor may be operated to detect the qualities of the targeted tissue, the gap distance, etc. before therapy, throughout therapy (simultaneously or alternating with therapy), after therapy, and/or any combination thereof.

The sensor is preferably one of several variations. In a first variation, the sensor is an ultrasound transducer, but may alternatively be any suitable sensor, such as an IR sensor or RF sensor, to detect the gap, the angle of the distal tip assembly 48, energy source 12, and/or sensor itself with respect to the tissue, the thickness of the tissue targeted for ablation, the characteristics of the ablated tissue, and any other suitable parameter or characteristic. The ultrasound transducer preferably utilizes a pulse of ultrasound of short duration, which is generally not sufficient for heating of the tissue. This is an ultrasound imaging technique, referred to in the art as A-Mode, or Amplitude Mode imaging. The sensor is preferably the same transducer as the transducer of the energy source, operating in a different mode (such as A-mode, described above), or may alternatively be a separate ultrasound transducer. The separate ultrasound transducer may be coupled to the transducer of the energy source 12 or may be in a separate location.

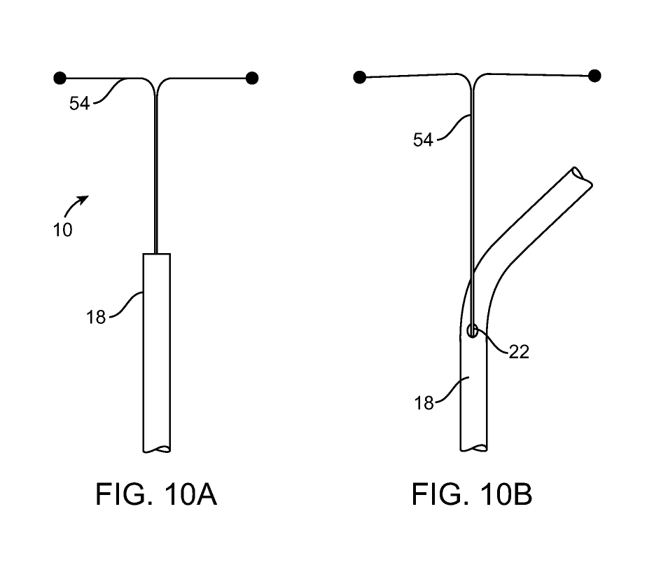

In a second variation, as shown in FIGS. 10A and 10B, the sensor is a positioning mechanism 54. The positioning mechanism is preferably coupled to a distal portion of the elongate member 18. In some variations, the positioning mechanism 54 is retractable into the elongate member 18. The positioning mechanism 54 functions to facilitate locating an anatomical structure by providing an indication of where the positioning mechanism 54 is with respect to the anatomical structure. The indication is preferably a visual indication (via a medical imaging system such as a fluoroscope), but is alternatively or additionally a tactile or audible indication. Additionally, the elongate member 18 and or the positioning mechanism 54 may include indicia, such as markings indicating distance, that indicate the location of the anatomical structure and/or to indicate the depth of insertion of the system 10 where the anatomical structure was located.

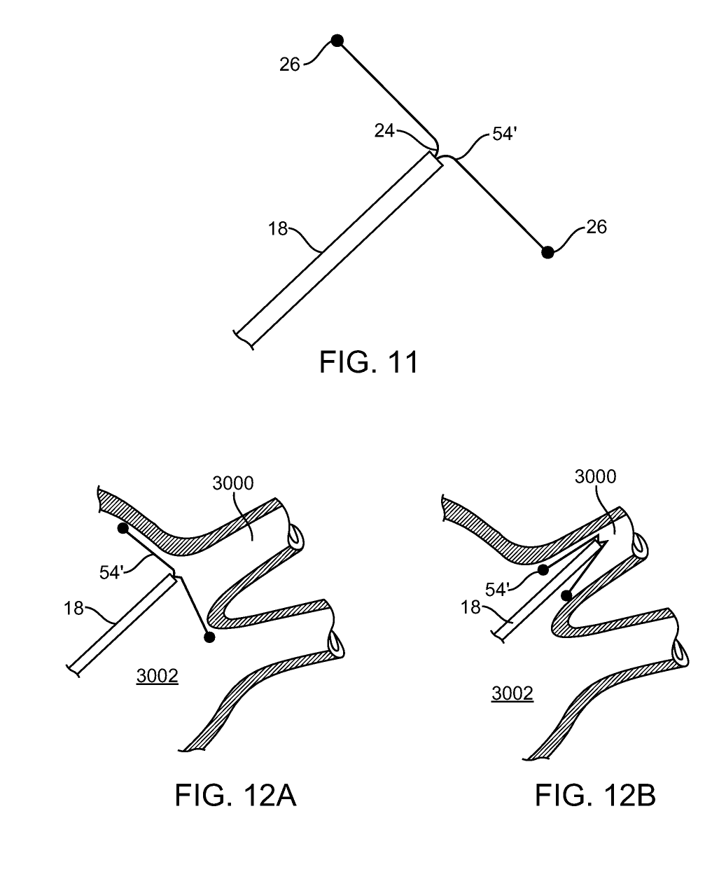

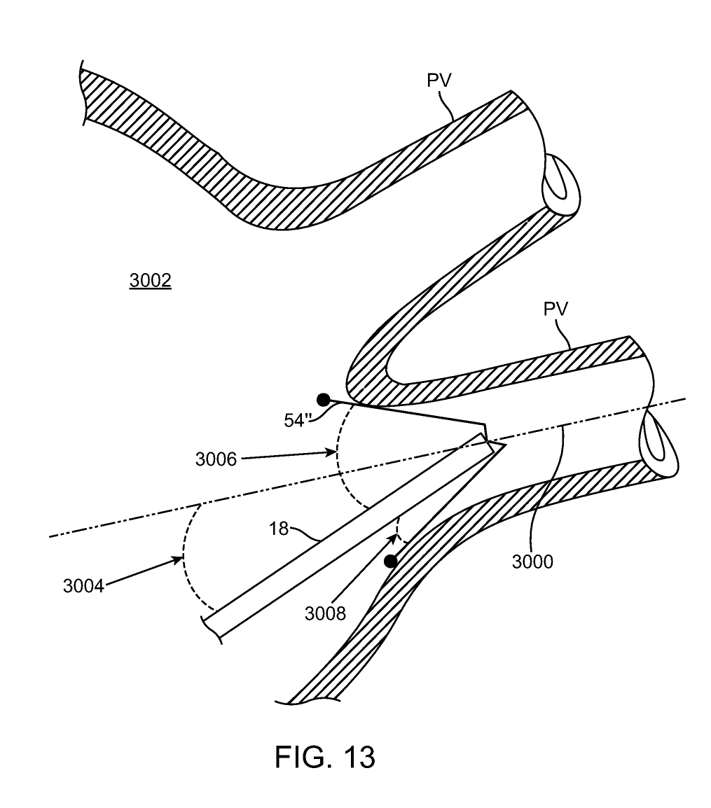

As shown in FIGS. 11, 12A, and 12B, a first version of the positioning mechanism 54′ includes a plurality of wires each having a first end 24 and a second end 26. The first end 24 is preferably coupled to the distal tip of the elongate member 18, but may alternatively be attached in any other suitable location. The second end 26 preferably extends from the distal tip of the elongate member and is positioned in a fully extended position, as shown in FIG. 11. The second end 26 preferably deflects due to contact with a surface, as shown in FIGS. 12A and 12B. The second end 26 is preferably biased towards the fully extended position, but may alternatively be biased towards any other suitable position.

As shown in FIGS. 12A and 12B, the plurality of wires function to facilitate locating an anatomical structure by flexing as they come in contact with the anatomical structure. For example, the wires will remain fully extended from the elongate member 18 when they are unobstructed in the left atrium of the heart 3002. As the system 10 is moved within the left atrium of the heart 3002 and begins to contact the ostium (opening) of a pulmonary vein 3000, the plurality of wires will begin to deflect partially, as shown in FIG. 12A. As the system 10 is moved into the pulmonary vein 3000, the wires will deflect more dramatically as shown in FIG. 12B. As the system is moved deeper into the pulmonary vein, the wires will not deflect as much, if at all, and the sensor and/or an operator of the system 10 will be able to determine when the positioning mechanism 54 of the system 10 is correctly located within the pulmonary vein. Furthermore, as shown in FIG. 13, the angle 3004 at which the system 10 enters the pulmonary vein 3000 with respect to the longitudinal axis of the pulmonary vein 3000 can be determined. Upon the detection of the angle 3004, the distal tip assembly 48 is preferably moved such that the angle between the energy source 12 and the tissue is an appropriate angle. The emitted energy beam 20 preferably contacts the target tissue at an angle between 20 and 160 degrees to the tissue, more preferably contacts the target tissue at an angle between 45 and 135 degrees to the tissue, and most preferably contacts the target tissue at an angle of 65 and 115 degrees to the tissue.

Positioning the System. FIGS. 10A-10B, 11, 12A-12B and 13 illustrate several embodiments of a positioning mechanism. The elongate member 18 and the distal tip assembly 48 cooperatively function to direct the energy source 12 and/or the sensor towards a target tissue. The elongate member 18 preferably moves and positions the distal tip assembly 48, and the energy source 12 and/or sensor within it. The distal tip assembly is preferably moved and positioned within a patient, preferably moved to within the left atrium of the heart (or into any other suitable location) and, once positioned there, is preferably moved to direct the sensor and/or the energy source 12 and the emitted energy beam 20 towards the target tissue at an appropriate angle. The emitted energy beam 20 preferably contacts the target tissue at an angle between 20 and 160 degrees to the tissue, more preferably contacts the target tissue at an angle between 45 and 135 degrees to the tissue, and most preferably contacts the target tissue at an angle of 65 and 115 degrees to the tissue.

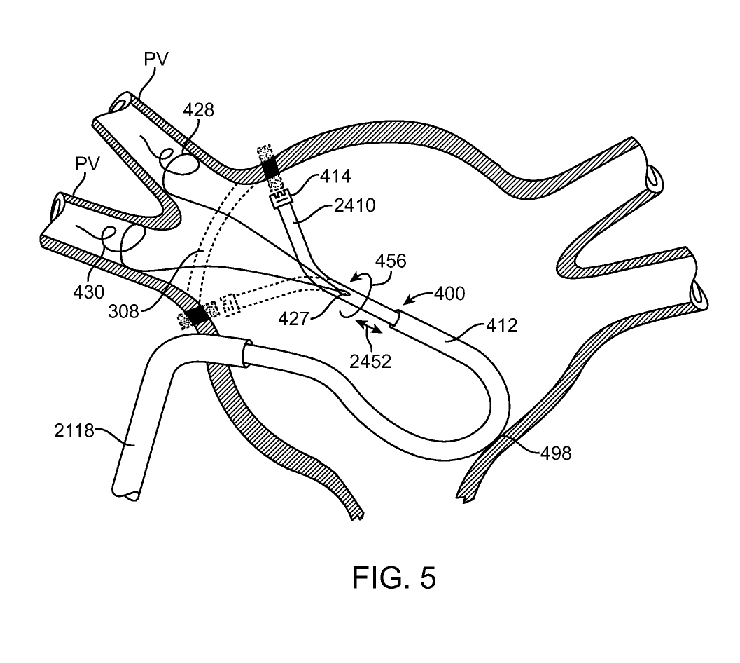

The distal tip assembly 48, and the energy source 12 (and/or sensor) within it, are preferably moved along an ablation path (and/or imaging path) such that the energy source 12 provides a partial or complete zone of ablation along the ablation path (and/or diagnosis of the tissue along the path). For example, as shown in FIG. 5, ablation path 308 encircles two pulmonary veins PV. The ablation path may alternatively have any suitable geometry and be positioned in any suitable location. The zone of ablation along the ablation path preferably has any suitable geometry to provide therapy, such as providing a conduction block for treatment of atrial fibrillation in a patient. The zone of ablation along the ablation path may alternatively provide any other suitable therapy for a patient. The imaging path preferably has any suitable geometry to assess the characteristics of the target tissue such as the gap (namely, the distance of the tissue surface from the energy source 12), the angle of the distal tip assembly 48, energy source 12, and/or sensor itself with respect to the tissue, the thickness of the tissue targeted for ablation, the characteristics of the ablated tissue, and any other suitable parameter or characteristic.

The elongate member 18 preferably moves and positions the distal tip assembly 48, and the energy source 12 and/or sensor within it, by moving in several variations of movements such as axial (forwards and backwards along the axis of the elongate member), rotational, and bending movements with several variations of mechanisms such as a bending mechanism and an anchoring mechanism in order to obtain the desired ablation and/or imaging paths. For example, a linear ablation path is preferably created by moving the distal tip assembly, and the energy source 12 within it, in bending movements. Additionally, a generally circular ablation path is preferably created by rotating the distal tip assembly, and the energy source 12 within it, about an axis and an elliptical path is created by a combination of bending and rotation movements.

Rotational Movement. As described above, and as shown in FIG. 1, the elongate member 18 and any of the catheters of the elongate member 18 are preferably rotated about the central axis of the elongate member as shown by arrows 2124 and 2156. Conventionally, as a catheter is rotated within a second catheter, the two catheters may bind and stick with one another, generally due to static friction, preventing a smooth rotational movement. To prevent and/or release this binding and/or torque buildup, the tubing of the catheters are preferably encased in a braid that is covered in a jacket of low friction material. The catheters may alternatively be encased in a spring, spring wrapping, or wrapping of foil. The material of the braid is preferably round or flat metal wires, plastic filaments, or Kevlar.

Alternatively to or in addition to the braid, the rotational movement of system 10 preferably includes a local oscillation movement (a second rotational movement in the opposite direction from the first rotational movement that is smaller than the first rotational movement). The local oscillation movement functions to prevent and/or release this binding and/or torque buildup. For example, as shown in FIG. 1, if rotational movement 2156 of the therapy catheter 2110 is clockwise a few degrees (or any other suitable distance), then the local oscillation movement (not shown) of the therapy catheter 2110 will be fewer degrees in the counter-clockwise direction. By taking a smaller rotational movement backward for every rotational movement forward, the binding and torque build up are minimized (and/or prevented), and the rotational movement of the catheter will preferably be substantially uniform and free of skipping and jumping.

The energy source 12 of the system 10 is uniquely suited for this local oscillation movement. For example, as the energy source 12 is energized and delivering the energy beam 20 (preferably ultrasound energy beam) to the target tissue and is rotated along an ablation path, the local oscillation movement (the back and forth movement) will bring the energy source 12 and the energy beam 20 over certain portions of the ablation path more than once. Due to the characteristics of the ultrasound energy beam and the lesion formation characteristics, the tissue will not be damaged or otherwise negatively affected by being contacted by the energy beam 20 more than once. This was verified in animal experiments.

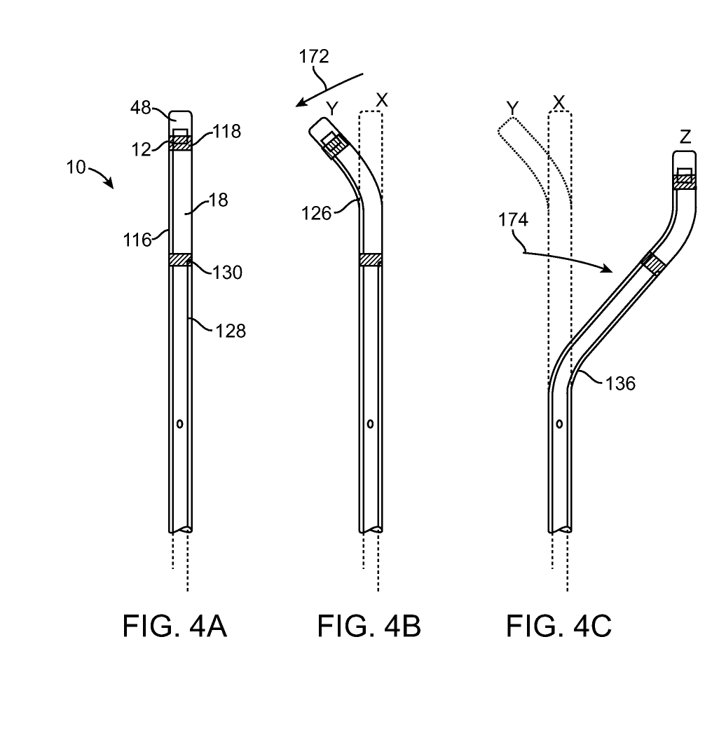

The Bending Mechanism and Bending Movement. As shown in FIGS. 4A-4C, the system 10 of the preferred embodiments further includes a bending mechanism that functions to bend a distal portion of the elongate member 18 in at least one of several locations. The bending mechanism preferably includes lengths of wires, ribbons, cables, lines, fibers, filament or any other tensional member. The bending mechanism is preferably one of several variations. In a first variation, as shown in FIGS. 4A-4C the bending mechanism preferably includes one or more pull wires, for example, a distal pull wire and a proximal pull wire that induce bending at a distal position and a proximal position. The distal pull wire and the proximal pull wire are preferably attached to the elongate member 18 with an adhesive band. Alternatively, the pull wires may be coupled to the elongate member 18 using any suitable attachment mechanism such as adhesive, welding, pins and/or screws. The pull wires are preferably disposed within one or separate lumens of the elongate member 18, but may alternatively be held in any suitable location. The pull wires preferably each terminate at a slider in a proximal housing that preferably includes various actuating mechanisms to affect various features of the bending mechanism 18. As shown in FIGS. 4A-4C, the distal pull wire 116 is secured at a distal portion of the elongate member 18 by means of the distal adhesive band 118. In use, as the distal pull wire 116 is pulled by moving a first slider (not shown), and the elongate member is bent at location 126 in the direction 172, thereby moving from position X to position Y, as shown in FIG. 4B. A proximal pull wire 128, which is secured in an elongate member lumen at a position by proximal adhesive band 130, is pulled by moving a second slider (not shown) and the elongate member bends at location 136 and moves in the direction 174 to position Z, away from the longitudinal axis of the catheter, as shown FIG. 4C. Alternatively, both pull wires may be pulled by a single slider mechanism and the shape illustrated in FIG. 4C can be achieved in this manner.

The pull wire attachment points, and correspondingly the bend locations in the device are preferably configurable, in any of a number of ways. For example, a single pull wire or other bend inducing mechanism may be used. Alternatively, the use of three or more such mechanisms may be used. With respect to attachment points for bend inducing mechanisms, any suitable location along the distal tip assembly as well as the catheter distal portion are suitable optional attachment points. With respect to the number and location of bend locations in the device, a spectrum of suitable bend locations may be provided. For example, while one and two bends are illustrated herein, three or more bends can be used to achieve a desired catheter configuration and/or application of energy using the device. The bending mechanism preferably includes any suitable number of bending locations (pivot points) such that elongate member is bendable into several variations of shapes and configurations. While the two bends of the bending mechanism may be in the same plane, as exemplified in FIG. 4A-4C, the bending mechanism preferably bends in any suitable plane and the two bends may occur in two different planes. The several variations of shapes and configurations preferably include shapes that allow the elongate member to position the distal tip assembly throughout an area of a patient (preferably throughout the left atrial chamber of the heart of a patient) and access target tissue within any section or portion of the chamber. For example, if the guide sheath were to enter through the septal wall of the left atrium, adjacent to (or close to) the ceiling wall of the left atrium, it would be beneficial to bend the elongate member down, away from the atrial ceiling and then back up towards the pulmonary veins. In a further example, in order to access the right pulmonary veins, which are typically closer to the entry point in the septal wall, a “shepherd's hook” configuration of the elongate member is beneficial wherein the elongate member enters the atrial chamber and then bends back towards the right pulmonary veins. As shown in FIG. 5, the outer catheter 412 has a preset shape of a “shepherd's hook” so as to point towards the right pulmonary veins when placed in the atrial chamber.

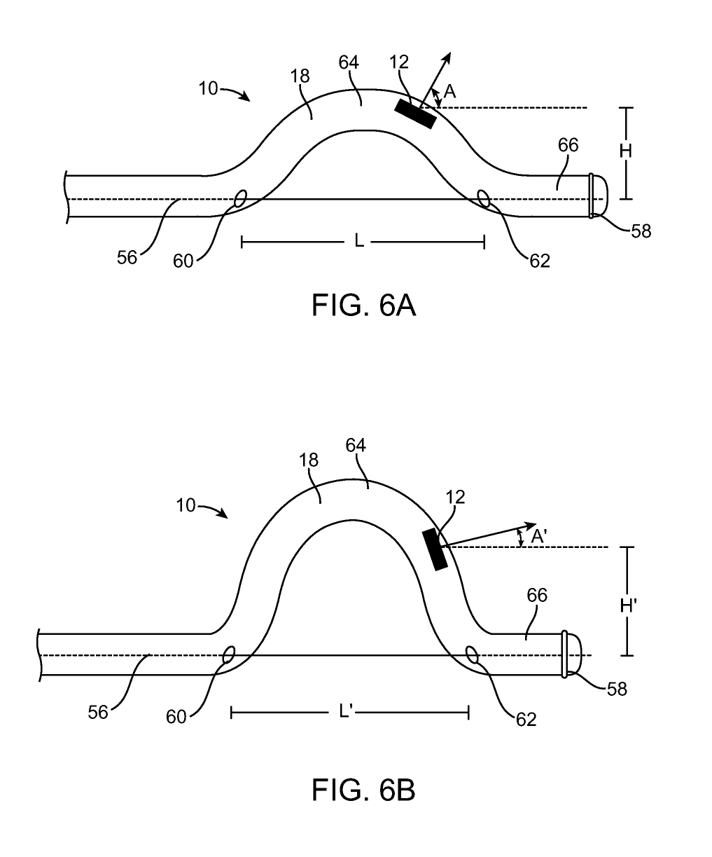

In a second variation, as shown in FIGS. 6A and 6B, the bending mechanism preferably includes at least one pull wire 56 that preferably induces “worm-like” bending in the elongate member. The pull wire is preferably attached to the elongate member 18 with an adhesive band 58. Alternatively, the pull wire may be coupled to the elongate member 18 using any suitable attachment mechanism such as adhesive, welding, pins and/or screws. The pull wire is preferably disposed within a lumen of the elongate member 18 and exits through notches 60 and 62, but may alternatively be held in any suitable location. The pull wire preferably terminates at a slider in a proximal housing (not shown) that preferably includes various actuating mechanisms to affect various features of the bending mechanism 18. In use, as the pull wire 56 is pulled by moving the first slider (not shown), and the elongate member is bent at location 64 while the distal tip portion 66 and the proximal portion of the elongate member remain unbent such that the center portion buckles and bends out about point 64, thereby moving the energy source 12 such that it moves away from the distal tip portion 66 (distance H) and to an angle A from the central axis of the elongate member 18. As the elongate member 18 is bent at point 64, the generally straight distal tip portion 66 and the proximal portion move to a distance L from one another. As shown in FIG. 6B, when the first slider (not shown) is moved further, the elongate member 18 will further buckle at location 64, thereby moving the energy source 12 such that it moves further away from the distal tip (distance H′, wherein distance H′ is greater than distance H) and to an angle A′ from the central axis of the elongate member. As the elongate member 18 is bent further at point 64, the generally straight distal tip portion 66 and the proximal portion move to a distance L′ from one another. Distance L′ is preferably shorter than distance L.

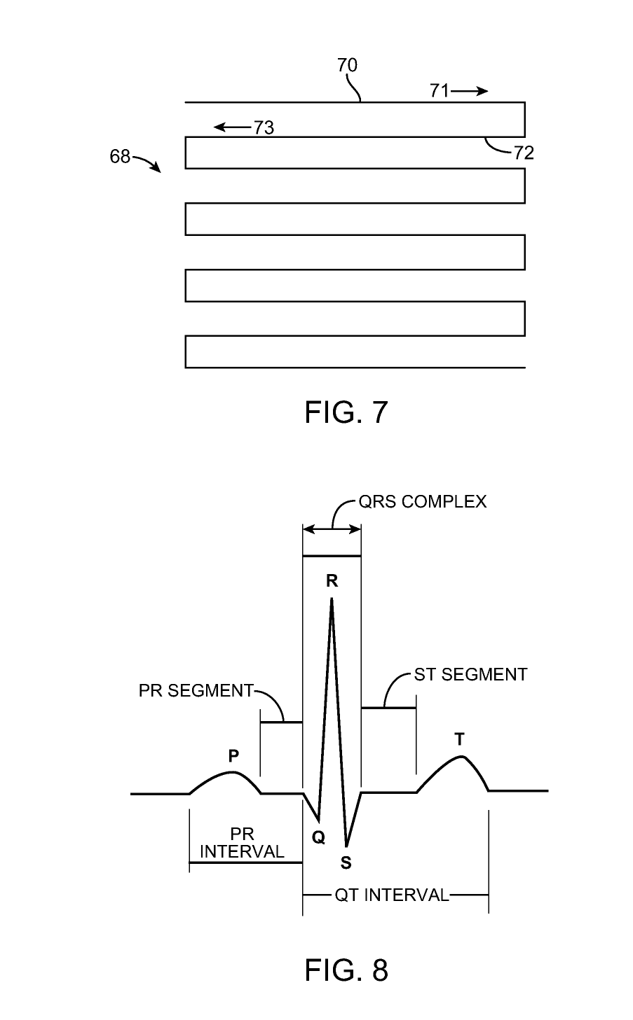

The bending mechanism functions to bend a distal portion of the elongate member 18 in at least one of several locations. Referring now to FIG. 7, the bending mechanism further functions to move the elongate member 18 through a series of bending movements such that the distal tip assembly 48, and the energy source and/or sensor, moves over a pattern 68 (such as an ablation path or a imaging path). The pattern is preferably one of several variations. In a first variation, as shown in FIG. 7, the pattern 68 is a raster pattern. The lines of FIG. 7 represent the path that the distal tip assembly, and the energy source and/or sensor within it, pass over as they are moved through the pattern 68 by the elongate member 18. The raster pattern is preferably formed by combining a series of left and right bends with a series of up and/or down bends. For example, as shown in FIG. 7, if the distal tip assembly 48 were to begin oriented towards the upper left hand corner of the pattern 68, the elongate member would first bend such that the distal tip assembly 48 moves towards the right, in a manner 71, and creates the first leg 70 of the pattern 68. The elongate member would then bend, preferably in the same location as before, such that the distal tip assembly moves down to begin the second leg 72 of the pattern 68 and from there the elongate member will bend such that the distal tip assembly 48 moves back towards the left, in a manner 73, and creates the second leg 72 of the pattern 68, and so on. The elongate member 18 can bend in any suitable location(s) and the distal tip assembly 48 can move in any suitable directions to move the energy source and/or the sensor back and forth such that it sweeps across the majority of an area, such as a wall or portion of a chamber of a heart.

The bending mechanism may further function to move the elongate member 18 through a series of bending movements such that the bending movements are synchronized to a movement of the patient, such as breathing, heart rate, or any other suitable movement. In this variation, the bending mechanism is preferably coupled to a heart rate monitor such as an electrocardiograph, which records the electrical activity of the heart over time. The electrical waves of the heart cause the heart muscle to pump, and therefore move in a generally predictable manner over time. By coupling the bending mechanism to the electrocardiograph, the bending mechanism can bend in such a way to accommodate for the movement of the heart, or the data received from the sensor during the movements through the pattern 68 can be altered to account for the movement of the heart. Furthermore, the data collected by the sensor during the movements through the pattern 68 can be gathered and/or displayed with respect to an Electrocardiogram (ECG or EKG)—a graphic that displays the overall rhythm of the heart produced by an electrocardiograph, as shown in FIG. 8. An ECG displays a series of tracings of the heartbeat (or cardiac cycle). Generally a single tracing, as shown in FIG. 8, will include a P wave, a QRS complex and a T wave. In a first version, the data collected by the sensor is preferably collected continuously and displayed with respect to an ECG. For example, the portion of the data displayed is preferably the portion of the data that was taken at the same point of each of the ECG tracings of the heartbeat (or the cardiac cycle). In a second version, the data collected by the sensor is preferably only collected once per cardiac cycle and preferably collected at the same point along the cardiac cycle. Although the sensor data is preferably collected and displayed in one of these two versions, the data may alternatively be collected and displayed in any other suitable fashion.

The Anchoring Mechanism. As shown in FIG. 3, the system 10 of the preferred embodiments further includes an anchoring mechanism that functions to hold the distal portion of the elongate member 18 in a relatively predictable position relative to a tissue, for example, inside a chamber such as the left atrium of the heart. The anchoring mechanism functions to provide a firm contact and/or stabilization between the anchor mechanism and the tissue, and provides an axis around which all or a portion of the catheter shaft can be rotated.

The anchoring mechanism is preferably one of several variations. As shown in FIG. 3, the anchor mechanism 570 of the ablation device includes a double wall tubing 580 having an annulus 582 between an inner wall 584 and an outer wall 586. Anchor mechanism 570 is an elongate structure spanning from a distal portion of the therapy catheter 510 to substantially the proximal portion of the device (not shown). The distal portion of the anchor mechanism 570 includes an expandable member 588, for example, an inflatable balloon, which can communicate with a connector, for example, a luer fitting (not shown) at the proximal end of the anchor mechanism 570. Although a balloon is described as an exemplary expandable member, it is envisioned that other expandable members such as a cage or stent may be used. The inner lumen 590 of the anchor mechanism 570 provides a passageway for the therapy catheter 510 such that the catheter is free to move axially 554 and rotationally 552 within. As shown in FIG. 3, during use, the anchor mechanism 570 can be positioned inside the guide sheath 522 and advanced distally until a distal portion of the anchor mechanism 570 extends beyond the guide sheath 522 while the expandable member 588 remains inside the guide sheath 522 substantially proximal to the guide sheath 522 end. In another implementation at least a part of the expandable member of the anchor mechanism remains inside the guide catheter, while another part of the expandable member extends distally beyond the guide catheter end (not shown). In yet another implementation the distal portion of the anchor mechanism remains substantially proximal to the distal end of the guide catheter (not shown).

To effect anchoring, the balloon can be inflated with a suitable fluid (e.g., saline or CO₂) sufficiently such that a distal portion of the anchor mechanism is held firmly in the guide catheter. The therapy catheter 510 can then be advanced distally (as shown by arrow 554 in FIG. 3) through the inner lumen 590 of the anchor 570. As shown in FIG. 3, when the expandable member 588 is inflated, the distal portion of the catheter 510 exiting from the anchor mechanism 570 is free to rotate in a manner 552 about a longitudinal axis, yet is held firmly in the guide sheath 522. As required, the catheter distal portion can be shaped by bending as described above to a desired position (as shown in FIGS. 4A-4C). When anchored at the end of the guide sheath 522, the distal portion of the therapy catheter 510 can be caused to follow a fixed rotational path without being susceptible to wavering or wandering as the catheter is rotated or otherwise guided in the heart chamber to create a zone of ablation.

In a second variation, the anchor mechanism includes a pre-shaped wire loop. The wire loop is preferably made of a shapeable wire, for example, made from a shape-memory material such as Nitinol (nickel-titanium alloy) and although a loop is described, it is envisioned that any of a number of shapes, curved and/or angular, two-dimensional and/or three-dimensional can provide the anchoring required. The anchor can reside in a lumen (not shown) of the elongate member 18, and can exit from the elongate member 18 through a notch near the distal end of the elongate member to couple with a tissue such that it anchors the system 10 with respect to the tissue. In a third variation, the expandable member is an inflatable balloon. The anchoring member may be in the shape of a disc that is inflatable, for example, an inflatable balloon. In a fourth variation, the distal portion of the anchor mechanism includes one or more barb members or similar tissue engaging hooks.

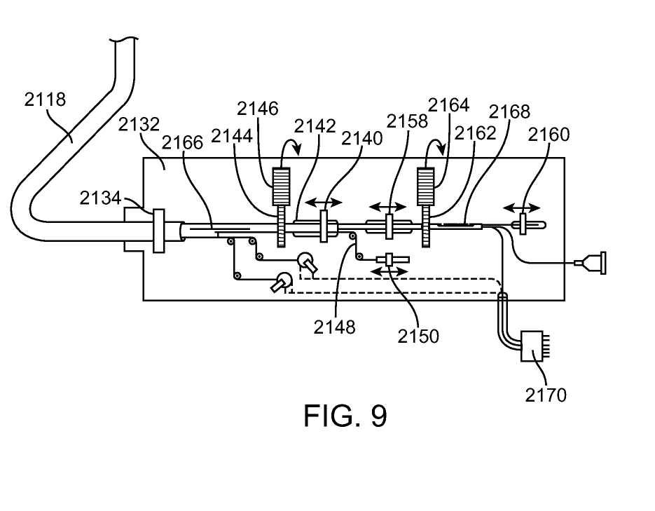

The Connector Console. As shown in FIG. 9, the system 10 of the preferred embodiments further includes a connector console 2132 that functions to move the elongate member 18 (guide sheath, outer catheter, therapy catheter, etc.) in any suitable combination of movements and to drive the various mechanisms of the system 10 (anchoring, bending, etc.). All of the movements described below may alternatively be achieved by hand or by using any other suitable motors, linkages, and actuators in the console 2132 or in a separate or additional driving device, and/or any combination thereof. As shown in FIG. 9, at the proximal end of the guide sheath 2118, the various catheter elements are connected to a variety of controls in a connector console 2132. After placement of the distal tip assembly 48 within the patient (in one example, preferably through the septum of the heart into the left atrium), the guide sheath 2118 is preferably locked in position by means of a lever 2134. As shown in FIG. 1 in conjunction with FIG. 9, the outer catheter 2112 is preferably provided with at least three independent movements: axial movement 2120, rotational movement 2124, and bending movement 2122. The axial movement 2120 of the outer catheter 2112 within the guide sheath 2118 is preferably achieved by moving slider 2140 that preferably moves linearly in slot 2142, but may alternatively be achieved in any other suitable fashion. Once the desired position of the catheter 2112 is achieved, the slider 2140 is preferably locked in position. The rotational movement 2124 of the outer catheter 2112 is preferably achieved by the gear mechanism 2144 and 2146, but may alternatively be achieved in any other suitable fashion. Gear 2144 is preferably attached to the proximal end of the outer catheter 2112. Gear 2144 is driven by the pinion 2146; which is attached to a motor (not shown). The bending movement 2122 around a pivot point 182 of the distal tip of the catheter 2112 is preferably achieved by means of the pull wire 2148, which terminates in a slider mechanism 2150, but may alternatively be achieved in any other suitable fashion. The slider 2150 is preferably lockable once the desired position of the bending of the catheter 2112 is achieved. Additionally, the outer catheter 2112 may bend in any suitable number of locations in addition to point 182.

Similarly to the outer catheter 2112, the therapy catheter 2110 also is provided with at least three independent movements: axial movement 2152, rotational movement 2156, and bending movement 2154. The catheter 2110 can be moved axially in the catheter 2112 as shown by movement 2152. This movement 2152 is preferably controlled at the proximal end by means of slider 2158, but may alternatively be achieved in any other suitable fashion. The slider 2158 is preferably lockable once the desired position of the therapy catheter 2110 is achieved in the outer catheter 2112. The distal portion of the catheter 2110 can be bent in the manner 2154 around a pivot point 184 preferably by a pull wire (not shown) connected to the slider mechanism 2160 at the proximal end console 2132, but may alternatively achieve the bending movement in any other suitable fashion. Again, the slider 2160 is preferably lockable in position once the desired position of the bend of the tip of the catheter 2110 is achieved. Additionally, the therapy catheter 2110 may bend in any suitable number of locations in addition to point 184. The catheter 2110 can be rotated in the outer catheter 2112 in a manner shown as 2156. The end portion 188 of the outer catheter 2112 preferably grips the catheter 2110 to provide support during the rotation 2156 of the catheter 2110 and the catheter 2110 is preferably freely movable inside the outer catheter 2112 in a manner 2152. This motion is preferably affected by the gear mechanism 2162 and 2164 in the console 2132, but may alternatively be effected by any other suitable mechanism. Gear 2162 is preferably attached to the proximal end of the catheter 2110, and it is preferably driven by the pinion 2164, which is connected to a motor (not shown). The catheters 2110 and 2112 preferably contain the corresponding orientation marks provided on the shafts thereof. The console also preferably includes a connector 2170, which electrically connects to a power generator and controller (not shown). The connector 2170 also provides electrical connections to the energy source 12 and/or the sensor of the distal tip assembly 48.

Method of Positioning the System. A method for positioning the energy source 12 within a patient and delivering energy to tissue, more specifically, for delivering ablation energy to tissue, such as heart tissue, to create a conduction for treatment of atrial fibrillation in a patient preferably includes the following steps. The method may alternatively include any other suitable steps or combinations thereof for any other suitable purpose and/or therapy.

A guide sheath is positioned across the atrial septum S of a heart in a conventional way (as shown in FIG. 1 and FIG. 5). The opening of the guide sheath is preferably directed towards the pulmonary veins of the heart chamber. In FIG. 5, the guide sheath 2118 is advanced across the atrial septum into the left atrium. The outer catheter 412 extends from the guide catheter 2118 and is bent into a curve 498 so that its distal tip faces toward a pulmonary vein. The therapeutic catheter 2410 having distal housing 414 extends from the outer catheter and may be slidably moved away from or into the outer catheter in the direction indicated by arrow 2452. The therapeutic catheter may also be rotated in the direction 456 (or in the opposite direction) relative to the outer catheter. The therapeutic catheter may also be bend along its length to further adjust the position of the distal assembly which directs energy into the tissue to be treated. In this embodiment, a plurality of coiled wires 428 extending from aperture 427 in the therapeutic catheter are positioned into the pulmonary veins in order to help anchor the device 400 relative to the treatment region. The distal assembly is thus rotated about the positioning wires 428, 430 to create a substantially circular zone of ablation around both pulmonary veins.

As shown in FIG. 3 (wherein the elongate member is positioned within the heart chamber as shown in FIG. 1), anchor mechanism 570 is advanced through the guide sheath 522 and beyond the guide sheath 522 open end towards the tissue area in the middle of the pulmonary veins (PV) (not shown) such that the anchor mechanism 522 points generally towards a part of the tissue surrounded by the PV.

Referring still to FIG. 3, the expandable member 588 of the anchor mechanism 570 is inflated with a fluid such that a distal portion of the anchor mechanism 570 is held firmly in the guide sheath 522.

The therapy catheter 510 is advanced through the inner lumen 590 of the anchor mechanism 570 and into the heart chamber.

As shown in FIGS. 4A-4C, the distal tip assembly 48 of the elongate member 18 is bent into a shape using the bending mechanism. In this step, the elongate member may be bent in any suitable configuration as described above, for any suitable purpose (e.g. for therapy and/or diagnosis).

Once within the heart chamber, the distal tip assembly 48 and the sensor within it are preferably moved around the chamber such that the sensor can detect the relevant characteristics of the tissue and location of the energy source 12 with respect to the tissue. The sensor is preferably moved along an imaging path that is the same as the intended ablation path and/or the sensor is preferably moved along an imaging sweep, as shown by pattern 68 in FIG. 7.

Once the system is positioned in the desired configuration and within the desired location with respect to the target tissue, and any desired imaging sweeps (one example of an imaging sweep is shown by pattern 68 in FIG. 7) have been performed, the energy source 12 is preferably energized by a generator (not shown) to provide an energy beam 20 of emitted ultrasound energy, which impinges on the target tissue. This energy beam 20 creates an ablation zone in the tissue along the desired (and verified) ablation path.

Referring again to FIG. 3, catheter 510 is progressively rotated about an axis in a manner 552 such that the tip assembly and the sound beam traverses in a substantially circular ablation path in the heart chamber. Alternatively, the catheter may be rotated and bent in a suitably combination to create a linear, or noncircular ablation path. The treatment of tissue along an ablation path is continued until a partial or a complete ablation of transmural thickness is achieved along the entire ablation path. As the therapy catheter 510 is rotated, the rotation movement preferably includes local oscillation movement, as described above, in order to reduce torque buildup and/or static friction such that the catheter does not jerk and will rotate smoothly.

As an added feature, the system can regularly, on a timeshared basis, convert from ablation mode briefly to imaging mode (e.g. convert from the activation of the energy source 12 to the sensor, or run both simultaneously). In this way, the correct gap or other parameters can be monitored during the ablation. A complete ablation ring is made around all the targeted pulmonary veins, thereby achieving a conduction block. As described, the ablation path is generally circular, but may alternatively be elliptical, linear, curved, and/or any suitable combination of geometries to preferably achieve a conduction block.

The elongate member is returned to a relaxed position by releasing the pull tension on the respective pull wires (not shown) and the therapy catheter 510 is retracted through the anchor mechanism.

The expandable member 588 of the anchor mechanism 570 is deflated and the anchor mechanism 570 is retracted through the guide sheath 522 and the guide sheath 522 is removed from the body.

Although omitted for conciseness, the preferred embodiments include every combination and permutation of the various elongate members 18, distal tip assemblies 48, energy sources 12, sensors, bending mechanisms, and anchoring mechanisms.

As a person skilled in the art will recognize from the previous detailed description and from the figures and claim, modifications and changes can be made to the preferred embodiments of the invention without departing from the scope of this invention defined in the following claims.

What is claimed is:

1. An ablation system for treating atrial fibrillation in a patient, said system comprising:
an elongate inner shaft having a proximal end, a distal end and a lumen therebetween;
a distal tip assembly adjacent the distal end of the inner shaft, the distal tip assembly comprising an energy source and a sensor, wherein the energy source is adapted to deliver energy to a target tissue so as to create a zone of ablation in the target tissue that blocks abnormal electrical activity thereby reducing or eliminating the atrial fibrillation in the patient; and
an elongate outer shaft having a proximal end, a distal end and a lumen therebetween,
wherein the inner shaft is slidably disposed in the outer shaft lumen, and wherein the inner shaft is rotatable, bendable and linearly slidable relative to the outer shaft, and wherein the outer shaft is rotatable, bendable and linearly slidable relative to the target tissue.

2. The system of claim 1, wherein the distal tip assembly comprises an outer housing, the energy source and sensor being disposed therein.

3. The system of claim 2, wherein the outer housing comprises an open end.

4. The system of claim 2, wherein the housing comprises a plurality of slots on one end forming a castellated region.

5. The system of claim 2, wherein the energy source is recessed from a distal end of the housing such that the energy source does not contact the target tissue when the energy source delivers energy.

6. The system of claim 1, wherein the energy source comprises an ultrasound transducer.

7. The system of claim 1, wherein the energy source delivers one of radiofrequency energy, microwaves, photonic energy, thermal energy, and cryogenic energy.

8. The system of claim 1, wherein the energy source delivers the energy at an angle ranging from 65 to 115 degrees relative to a surface of the target tissue.

9. The system of claim 1, wherein the zone of ablation follows an arcuate path.

10. The system of claim 1, wherein the zone of ablation follows a linear path.

11. The system of claim 1, wherein the sensor is adapted to detect gap distance between a surface of the target tissue and the energy source.

12. The system of claim 1, wherein the sensor is adapted to detect an angle between the energy source and a surface of the target tissue.

13. The system of claim 1, wherein the sensor is adapted to determine thickness of the target tissue.

14. The system of claim 1, wherein the sensor is adapted to determine characteristics of the target tissue.

15. The system of claim 1, wherein the sensor comprises an ultrasound transducer.

16. The system of claim 15, wherein the energy source comprises the same ultrasound transducer.

17. The system of claim 1, wherein the sensor comprises one of an infrared sensor and a radiofrequency sensor.

18. The system of claim 1, wherein the sensor comprises a positioning mechanism adjacent the distal end of the outer shaft, wherein the positioning mechanism is adapted to facilitate location of an anatomic structure and also adapted to anchor the ablation system to the anatomic structure.

19. The system of claim 18, wherein the inner shaft is rotatable around the positioning mechanism.

20. The system of claim 18, wherein the positioning mechanism is positionable in the outer shaft lumen, and wherein the positioning mechanism is in a substantially linear configuration while disposed therein.

21. The system of claim 18, wherein the positioning mechanism comprises a coil.

22. The system of claim 18, wherein the positioning mechanism comprises a plurality of wires biased to flare radially outward when unconstrained.

23. The system of claim 18, wherein the positioning mechanism is adapted to exert an outward biasing force against the anatomic structure, thereby anchoring the ablation system thereto.

24. The system of claim 18, wherein the anatomic structure comprises a pulmonary vein, and wherein the positioning mechanism is positionable in the pulmonary vein and adapted to indicate an angle of entry of the inner shaft into the pulmonary vein.

25. The system of claim 1, further comprising a guide catheter, the outer shaft being slidably positioned in the guide catheter.

26. The system of claim 1, wherein the target tissue comprises heart tissue.

27. The system of claim 1, wherein the target tissue comprises a pulmonary vein or tissue adjacent thereto.

28. The system of claim 1, wherein the inner shaft or the outer shaft comprises a braided portion.

29. The system of claim 1, wherein the inner shaft or the outer shaft comprises a spring coil.

30. The system of claim 25, further comprising an anchoring mechanism coupled with the ablation system and configured to stabilize the distal tip assembly.

31. The system of claim 30, wherein the anchor mechanism comprises an expandable member.

32. The system of claim 31, wherein the expandable member comprises a balloon.

33. The system of claim 30, wherein the anchor member comprises a shapeable wire coupleable with the target tissue.

34. The system of claim 30, wherein the anchor member comprises one or more tissue engaging barbs or hooks.

35. The system of claim 1, further comprising a bending mechanism operably coupled with the inner shaft.

36. The system of claim 35, wherein the bending mechanism comprises a pull wire operably coupled adjacent the distal end of the inner shaft and wherein a portion of the pull wire is disposed along an outer surface of the inner shaft such that when the pull wire is actuated, the inner shaft deflects radially inward or outward relative to the portion of the pull wire outside of the inner shaft, and wherein the portion of the pull wire remains in a substantially linear configuration.

37. The system of claim 35, wherein the bending mechanism comprises a first and a second pull wire, the first pull wire coupled with a distal region of the inner shaft and the second pull wire coupled with a proximal region of the inner shaft, the pull wires adapted to bend the inner shaft in two locations, a first bend and a second bend.

38. The system of claim 37, further comprising an actuator disposed near a proximal end of the inner shaft and adapted to actuate the pull wires thereby bending the inner shaft and forming the first bend and the second bend along the inner shaft.

39. The system of claim 38, wherein the first bend and the second bend are in different planes.

40. The system of claim 1, further comprising a bending mechanism operably coupled with the outer shaft.

41. The system of claim 40, wherein the bending mechanism comprises a first and a second pull wire, the first pull wire coupled with a distal region of the outer shaft and the second pull wire coupled with a proximal region of the outer shaft, the pull wires adapted to bend the outer shaft in two locations, a first bend and a second bend.

42. The system of claim 41, further comprising an actuator disposed near a proximal end of the outer shaft and adapted to actuate the pull wires thereby bending the inner shaft and forming the first bend and the second bend along the inner shaft.

43. The system of claim 42, wherein the first bend and the second bend are in different planes.

44. A method for treating atrial fibrillation in a patient by ablating tissue, said method comprising:
providing an ablation system comprising an outer shaft and an inner shaft having a distal tip assembly, the distal tip assembly comprising an energy source and a sensor, wherein the outer shaft is slidably disposed over at least a portion of the inner shaft;
positioning the distal tip assembly adjacent the tissue;
manipulating the inner shaft or the outer shaft so as to place the energy source in a desired position relative to the tissue;
delivering energy from the energy source to the tissue; and
creating a partial or complete zone of ablation in the tissue thereby blocking abnormal electrical activity and reducing or eliminating the atrial fibrillation.

45. The method of claim 44, wherein the ablation system further comprises a guide sheath, the method further comprising positioning a distal portion of the guide sheath across an atrial septum of the patient's heart.

46. The method of claim 44, wherein the step of positioning comprises advancing the distal tip assembly intravascularly into the patient's heart.

47. The method of claim 44, wherein the step of manipulating comprises slidably moving the inner shaft relative to the outer shaft.

48. The method of claim 44, wherein the step of manipulating comprises rotating the inner shaft relative to the outer shaft.

49. The method of claim 44, wherein the step of manipulating comprises bending the inner shaft.

50. The method of claim 49, wherein the bending comprises bending the inner shaft in two or more locations.

51. The method of claim 50, wherein the two or more bends lie in the same plane.

52. The method of claim 49, wherein the bending comprises actuating one or more pull wires coupled to the inner shaft.

53. The method of claim 44, wherein the step of manipulating comprises slidably moving the outer shaft.

54. The method of claim 44, wherein the step of manipulating comprises rotating the outer shaft.

55. The method of claim 44, wherein the step of manipulating comprises bending the outer shaft.

56. The method of claim 55, wherein the bending comprises bending the outer shaft in two or more locations.

57. The method of claim 56, wherein the two or more bends lie in the same plane.

58. The method of claim 55, wherein the bending comprises actuating one or more pull wires coupled to the outer shaft.

59. The method of claim 44, wherein the step of manipulating comprises rotating either the inner or the outer shaft in a first direction and rotating the inner or the outer shaft in a second direction opposite the first direction so as to reduce binding or torque buildup in the inner or the outer shaft.

60. The method of claim 44, wherein the step of manipulating comprises moving the energy source so as to direct the energy from the energy source to the tissue in a raster pattern.

61. The method of claim 44, wherein the step of manipulating comprises synchronizing movement of the energy source with the patient's heart rate.

62. The method of claim 44, wherein the energy source comprises an ultrasound transducer and the step of delivering the energy comprises delivering an ultrasound beam from the transducer to the tissue.

63. The method of claim 44, wherein the step of delivering the energy comprises delivering one of radiofrequency energy, microwave energy, photonic energy, thermal energy, and cryogenic energy.

64. The method of claim 44, wherein the step of creating the zone of ablation comprises forming a circular ablation path.

65. The method of claim 64, wherein the ablation path encircles at least one pulmonary vein.

66. The method of claim 44, wherein the step of creating the zone of ablation comprises forming a linear ablation path.

67. The method of claim 44, wherein the ablation system further comprises a sensor, the method further comprising sensing characteristics of the tissue with the sensor.

68. The method of claim 67, wherein the sensor comprises an ultrasound transducer.

69. The method of claim 68, wherein the energy source comprises the same ultrasound transducer.

70. The method of claim 69, further comprising switching modes between delivering energy from the ultrasound transducer and sensing with the ultrasound transducer.

71. The method of claim 67, wherein the characteristics of the tissue comprise position of the tissue relative to the energy source.

72. The method of claim 71, wherein the position comprises a gap distance between the tissue and a surface of the energy source.

73. The method of claim 71, wherein the position comprises a relative angle between the energy source and the tissue.

74. The method of claim 71, wherein the characteristics of the tissue comprise thickness of the tissue or thickness of the ablation zone.

75. The method of claim 67, wherein the sensor comprises a positioning mechanism, the method further comprising advancing the positioning mechanism from either the inner or the outer shaft into the tissue or tissue adjacent thereto, the positioning mechanism facilitating locating an anatomical structure.

76. The method of claim 75, wherein the positioning mechanism comprises a plurality of wires, the method further comprising positioning the wires into a pulmonary vein while observing the shape of the wires.

77. The method of claim 71, further comprising guiding the energy based on the characteristics that are sensed.

78. The method of claim 72, further comprising maintaining the gap at a desired value.

79. The method of claim 44, wherein the tissue comprises left atrial tissue.

80. The method of claim 44, wherein the tissue comprises a pulmonary vein or tissue adjacent thereto.

81. The method of claim 44, further comprising cooling the energy source.

82. The method of claim 44, further comprising anchoring the distal tip assembly relative to the tissue.

83. The method of claim 82, wherein the anchoring step comprises coupling a wire with the tissue.

84. The method of claim 82, wherein the anchoring step comprises expanding an expandable member disposed on the outer shaft.

85. The method of claim 84, wherein the expandable member comprises a balloon.
